(19) United States
(12) Patent Application Publication
(10) Pub. No.: US 2025/0232690 A1
(43) Pub. Date: Jul. 17, 2025

(54) MOTION SENSITIVE LED GLASSES

(71) Applicant: KING FAISAL UNIVERSITY, AL-AHSA (SA)

(72) Inventors: RIHAM ABDULRAHMAN ALJAFARI, AL-AHSA (SA); ABDULRAHMAN ALSAYED, AL-AHSA (SA); ABDULLHAMID ALARFAJ, AL-AHSA (SA)

(21) Appl. No.: 18/414,304

(22) Filed: Jan. 16, 2024

(51) Int. Cl. G09B 19/00; G02C 11/04; G08B 5/36; H05B 47/115

(52) U.S. Cl. CPC G09B 19/00; G02C 11/04; H05B 47/115; G08B 5/36

(57) ABSTRACT

Motion sensitive eyeglasses include a frame; a motion sensor located along the frame; and LEDs located along the frame. The LEDs are illuminated when motion is detected by the motion sensor.

### BACKGROUND

### 1. Field

The present disclosure relates to systems that assist in identifying unwanted behavior, and particularly to an eye glass system that identifies unwanted behavior.

### 2. Description of the Related Art

Teachers in the classroom face inappropriate behavior from students in many circumstances. This unwanted behavior affects the classrooms ability to gain knowledge directly and indirectly. Students with disabilities are more likely to have unacceptable behavior compared to students in general education. Controlling behavior in these students is important because students with disabilities may be included in the general education classroom. Therefore, it is necessary to have a device that helps general education teachers identify unwanted behavior and provide immediate intervention.

### SUMMARY

Eyeglasses that can be worn by a student who may engage in distractive behavior in a classroom setting tracks the motion of the head of the student. If the head of the student is determined to be in a position which shows evidence of not paying attention, an LED light turns red alerting both the student and teacher of the undesired behavior. If the head movement returns to a more attentive state, then the LED light turns yellow. The LED turns green after a prolonged determination of attentiveness.

Motion sensitive eyeglasses, in one embodiment, include a frame; a motion sensor located along the frame; and LEDs located along the frame. The LEDs are illuminated when motion is detected by the motion sensor.

The LEDs include red LEDs, yellow LEDs and green LEDs.

The red LEDs are illuminated when the motion sensor detects motion.

The yellow LEDs are illuminated when the motion sensor detects a decrease in motion.

The green LEDs are illuminated when the motion sensor does not detect motion.

A method for utilitizing motion sensitive eyeglasses includes sensing motion of motion sensitive eyeglasses through a motion sensor located along a frame of the motion sensitive eyeglasses; and illuminating red LEDs located along the frame of the motion sensitive eyeglasses when motion is sensed by the motion sensor.

The method further includes illuminating yellow LEDs located along the frame of the motion sensitive eyeglasses when decreased motion is detected by the motion sensor.

Green LEDs located along the frame of the motion sensitive eyeglasses are illuminated when no motion is detected by the motion sensor.

These and other features of the present subject matter will become readily apparent upon further review of the following specification.

### BRIEF DESCRIPTION OF THE DRAWINGS

FIG. 1 is an illustration of a user wearing glasses in normal mode.

FIG. 2 is an illustration of the user wearing glasses in an active unattentive mode.

FIG. 3 is an illustration of the user wearing glasses in a positive response mode.

FIG. 4 is an illustration of the user wearing glasses in an attentive mode.

FIG. 5 is an illustration of the glasses with LEDs and motion sensors.

Similar reference characters denote corresponding features consistently throughout the attached drawings.

### DETAILED DESCRIPTION OF THE PREFERRED EMBODIMENTS

Motion sensitive eyeglasses can be worn by students who may engage in distractive behavior in a classroom setting. The eyeglasses can include a Light Emitting Diode (LED) light extending along a rim of the glasses and a sensor for detecting any head movement of the wearer of the eyeglasses. The LED light is configured to display one of three colors, e.g., green, red, and yellow, depending upon a movement or movement frequency of the wearer's head. For example, when a student is quiet and has little or no motion, the sensor can send a signal to cause the LED to turn green. When a student is active and moves their head frequently, the sensor can send a signal to cause the LED to turn red. When the head motion stops, the sensor can send a signal to cause the LED to turn yellow. The green light can be activated every 5 or 10 minutes depending on an adjustment that made by the teacher. When the LED is red, the teacher can easily identify misbehaving students and help them get back on task. Students themselves can also be alerted to their misbehavior when they see the red light.

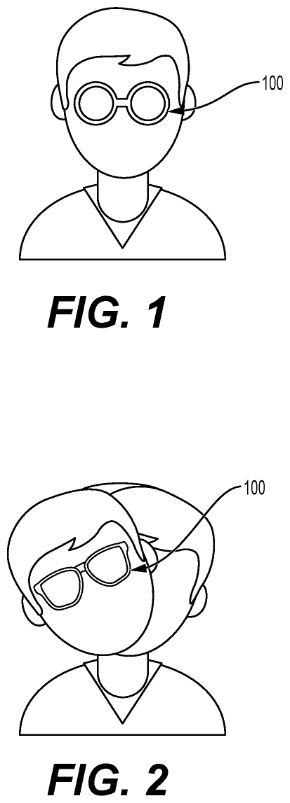

FIG. 1 is an illustration of a user wearing motion sensitive eyeglasses 100 in normal mode. In this mode the user is attentive and listening.

FIG. 2 is an illustration of a user wearing motion sensitive eyeglasses 100 in an active, unattentive mode. In this illustration, the user is moving their head back and forth. Sensors within the motion sensitive eyeglasses 100 indicate that there is active movement and that the user is not being attentive. The motion sensitive eyeglasses 100 illuminate the red LEDs within the frame of the motion sensitive eyeglasses 100 to indicate that the user is not being attentive based on the frequency and/or duration of the head motion. The illuminated red LEDs are visible to a third party, such as a teacher, who can then intervene to get the user to become attentive again. It is also possible that the user can see the illuminated red LEDs and take corrective measures on their own to become attentive again

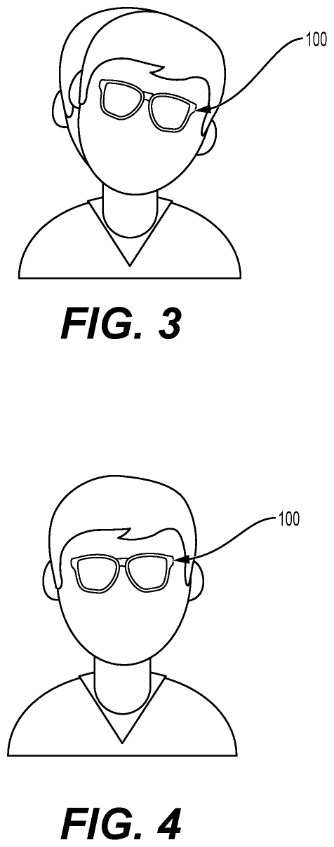

FIG. 3 is an illustration of a user wearing motion sensitive eyeglasses 100 in a positive response mode. In this mode the user is made aware that they are not being attentive and adjusts their behavior to become more attentive. In this figure, the user slows down the movement of their head. The yellow LEDs are illuminated within the frame in this figure indicating that the user is getting back on track to being attentive again because the sensors have sensed a decrease in movement of the head after having sensed movement of the head sufficient to illuminate the red LEDs. The decrease in movement is in comparison with the amount of movement the sensors sense to activate the red LEDs.

Once the user gets back on track and is attentive, the green LEDs in the frame of the motion sensitive eyeglasses 100 are illuminated, as shown in FIG. 4. The illumination of the green LEDs indicate that the user is attentive and paying attention. The LEDs turn green (from yellow) when the user has been alert and attentative for a given period of time, namely from 1 to 5 minutes of continuous attentativeness. A teacher is able to adjust the period of attentativeness needed to change the color from yellow to green, or the eyeglasses could be programmed to have the green LEDs lit after any given period of attentativeness.

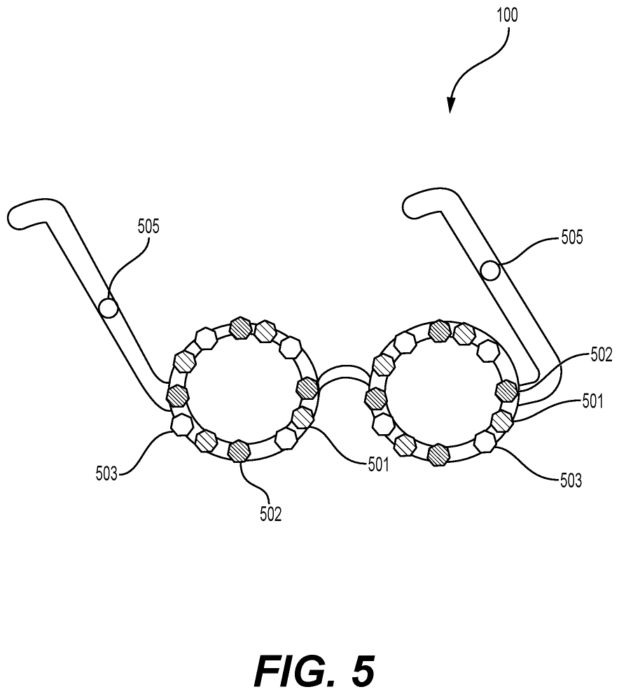

FIG. 5 is an illustration of the motion sensitive eyeglasses 100 with LEDs and motion sensors. They include LEDs in the frame of the glasses. In this figure there are three types of LEDs, green LEDs 501, yellow LEDs 502 and red LEDs 503. Motion sensors 505 can determine movement of the user's head.

The motion sensitive eyeglasses 100 can be useful for students with disabilities. This includes students with Intellectual Disabilities, students who are Hard of Hearing, students with Cochlear Implants, students with Autism, students with Emotional and Behavioral Disorders, students with Attention Deficit Hyperactivity Disorder. Also, General education students may use this device if needed.

It is to be understood that the present subject matter is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. Motion sensitive eyeglasses, comprising:
a frame;
a motion sensor located along the frame; and
LEDs located along the frame, wherein the LEDs are illuminated when motion is detected by the motion sensor.

2. The motion sensitive eyeglasses as recited in claim 1, wherein the LEDs include red LEDs, yellow LEDs and green LEDs.

3. The motion sensitive eyeglasses as recited in claim 2, wherein the red LEDs are illuminated when the motion sensor detects motion.

4. The motion sensitive eyeglasses as recited in claim 2, wherein the yellow LEDs are illuminated when the motion sensor detects a decrease in motion.

5. The motion sensitive eyeglasses as recited in claim 2, wherein the green LEDs are illuminated when the motion sensor does not detect motion.

6. A method of utilizing motion sensitive eyeglasses, comprising:
sensing motion of motion sensitive eyeglasses through a motion sensor located along a frame of the motion sensitive eyeglasses; and
illuminating red LEDs located along the frame of the motion sensitive eyeglasses when motion is sensed by the motion sensor.

7. The method as recited in claim 6, further comprising illuminating yellow LEDs located along the frame of the motion sensitive eyeglasses when decreased motion is detected by the motion sensor.

8. The method as recited in claim 6, further comprising illuminating green LEDs located along the frame of the motion sensitive eyeglasses when no motion is detected by the motion sensor.
